(19) United States
(12) Patent Application Publication
(10) Pub. No.: US 2024/0238490 A1
(43) Pub. Date: Jul. 18, 2024

(54) DIALYSIS MACHINE

(71) Applicant: FRESENIUS MEDICAL CARE DEUTSCHLAND GMBH, Bad Homburg (DE)

(72) Inventors: Thorsten HOCHREIN, Eschenau/Knetzgau (DE); Frank HEDMANN, VOLKACH (DE); Nico NIEDERMEYER, Bergrheinfeld (DE); Stefan SAAL, Abersfeld (DE); Simone LIPPERT, Gemünden am Main (DE)

(73) Assignee: FRESENIUS MEDICAL CARE DEUTSCHLAND GMBH, Bad Homburg (DE)

(21) Appl. No.: 18/558,792

(22) PCT Filed: May 4, 2022
(86) PCT No.: PCT/EP2022/062049

§ 371 Date: Nov. 3, 2023

(30) Foreign Application Priority Data
May 5, 2021 (DE) 10 2021 111 685.6

(51) Int. Cl. A61M 1/14

(52) U.S. Cl. CPC A61M 1/152

(57) ABSTRACT

The present invention relates to a blood treatment device, preferably a dialysis machine, that is configured to be used with a disposable, preferably with a cassette system, and that has a coupling region for the disposable and a control unit that is configured to carry out a plurality of pressure measurements at different times during a coupling procedure of the disposable to the blood treatment device, with a quality of the coupling of the disposable to the blood treatment device being able to be determined with reference thereto. The present invention further relates to a method of monitoring a coupling procedure of a disposable to a blood treatment device.

The present invention relates to a blood treatment device that is configured to determine the quality of the coupling of a disposable component (disposable) to the blood treatment device with reference to a pressure measurement profile during a setup phase or during a coupling procedure of the disposable.to the blood treatment device. The invention furthermore relates to a corresponding method.

On the use of blood treatment devices, in particular of dialysis machines, it is essential for the ensuring of patient safety to ensure that a correct disposable is correctly coupled to the blood treatment device. Disposables not completely connected or also disposables incorrectly used multiple times can endanger the safety of the patient.

Ensuring a correctly performed coupling procedure in particular plays an important role in the field of peritoneal dialysis since the patients usually set up the dialysis machine for the treatment independently at home and thus also couple the disposables independently.

It is thus the underlying object of the present invention against this background to alleviate or even to fully eliminate the problems of the prior art. It is in particular the underlying object of the present invention to provide a blood treatment device by means of which a coupling procedure of a disposable to the blood treatment device can be monitored.

This object is achieved by a blood treatment device in accordance with claim 1 and by a method in accordance with claim 10. Advantageous further developments of the invention form the subject of the dependent claims.

A blood treatment device, preferably a dialysis machine, is accordingly provided that is configured to be used with a disposable, preferably with a cassette system and that has a coupling region for the disposable and a control unit that is configured to carry out a plurality of pressure measurements at different times during a coupling procedure of the disposable to the blood treatment device, with a quality of the coupling of the disposable to the blood treatment device being able to be determined with reference thereto.

A blood treatment device in accordance with the invention can, for example, be a device for peritoneal dialysis. The use of the present invention in hemodialysis is therefore also conceivable.

A cassette system, preferably a pressed cassette system, is preferably used as the disposable and has at least one flexible membrane and a possibility of pressure measurement. Both cassettes having a hard part on one side and a flexible membrane on the oppositely disposed side or cassettes having a hard part as a frame or core that is equipped with a flexible membrane at both sides can be used.

A blood treatment device in accordance with the invention is preferably furthermore equipped with an evaluation unit that is configured to evaluate a time sequence of a pressure profile prepared on the basis on the plurality of pressure measurements, preferably with respect to a pressure level and/or to a pressure change rate and to determine the quality of the coupling of the disposable to the blood treatment device on the basis thereof.

For a further improvement of the precision of the evaluation, environmental conditions and/or properties of the disposable, in particular material properties and/or tolerances can be taken into account in the determination of the quality of the coupling of the disposable to the blood treatment device.

In accordance with an embodiment, in accordance with the invention, the blood treatment device has a pressure measurement device that is suitable or configured to record or to detect a pressure in the disposable, in particular at least one pump chamber of the cassette. In this respect, the pressure measurement device can e.g. be coupled to the disposable or be a component of the disposable.

In accordance with a further embodiment in accordance with the invention, the blood treatment device has a pump system that preferably comprises one or more pumps and a pressure measurement device, with the plurality of pressure measurements within the pump system preferably taking place at or in the pump system or at or in the one or more pumps.

At least two pumps can be provided, for example, and the pressure measurement takes place at or in these two pumps. A pressure measurement is also conceivable at a line system that can be arranged between the pump and the disposable, in particular a supply line to at least one pump chamber of the cassette.

The application is here independent of the pump method (membrane pump, piston pump, hose roller pump, etc.). The invention is thus also not restricted to a specific pump type.

Any desired number of pressure measurements can generally be carried out at different times during the coupling procedure. It has proven advantageous in practice for the control device to be configured to carry out a pressure measurement at at least a first time after the disposable has been connected to the blood treatment device and before the disposable has been flushed and/or at a second time after the disposable has been installed on the blood treatment device and after the disposable has been flushed.

The pressure can, for example, be measured in two different pumps in each case before the flushing and after the flushing of the disposable.

Provision can furthermore be made in accordance with the invention that the evaluation unit is configured to compare the pressure measurement values determined for a plurality of pumps or their time sequences with one another respectively to determine the quality of a coordination of the pumps.

For example, the detected pressure values of the two different pumps can each be compared with one another before the flushing and after the flushing of the disposable to check whether the pumps have the same or substantially the same pressure. In this respect, a threshold value of a maximum permitted deviation between the pressure measurement values of the pumps, e.g. of 100 mbar, can be provided.

In general, the evaluation unit can be configured to compare the determined pressure measurement values and/or their time sequences in each case with a threshold value and/or with a tolerance range surrounding it and the blood treatment device can preferably furthermore be configured to actuate a corresponding output as to whether the pressure measurement values or their time sequences are in a desired ratio with the respective threshold value and/or tolerance range.

A blood treatment device in accordance with the invention can furthermore be configured to automatically electronically register and document the detected pressure measurement values and the data prepared by the evaluation unit. These data are used, for example, as part of predictive maintenance of the blood treatment device or also to document and ensure patient safety.

The blood treatment device can furthermore be configured to actuate an output with respect to the determined quality of the coupling of the disposable to the blood treatment device and/or to only release an operation of the blood treatment device when a certain quality of the coupling of the disposable to the blood treatment device exceeds a predetermined threshold value. The output can take place optically and/or acoustically.

A further aspect of the invention relates to a method of determining a quality of the coupling of a disposable, in particular a cassette system, to a blood treatment device, in particular a dialysis machine, the methods comprising the steps:
carrying out a plurality of pressure measurements at different times during a coupling procedure of the disposable to the blood treatment device to determine pressure measurement values; and
evaluating a time sequence of a pressure profile prepared on the basis of the plurality of pressure measurements, preferably with respect to at least one pressure level and/or a pressure change rate and determining the quality of the coupling of the disposable to the blood treatment device on the basis thereof.

A method in accordance with the invention can preferably have all or also only certain ones of the features that were described above with reference to a blood treatment device in accordance with the invention and that will not be reproduced in full here. A method in accordance with the invention can be used on a blood treatment device in accordance with the invention.

Provision can in particular be made within the framework of a method in accordance with the invention that a pressure measurement is at least carried out at a first time after the disposable has been mounted on the blood treatment device and before the disposable has been flushed and/or at a second time after the disposable has been installed on the blood treatment device and after the disposable has been flushed.

The plurality of pressure measurements can furthermore take place at or in the one or more pumps of a blood treatment device, preferably a blood treatment device in accordance with the invention

Provision can furthermore be made with a method in accordance with the invention that pressure measurement values are determined for a plurality of pumps and that the pressure measurement values or their time sequences are compared with one another to determine the quality of a coordination of the pumps.

A method in accordance with the invention can furthermore generally comprise the step that the determined pressure measurement values or their time sequences are respectively compared with a threshold value and/or with a tolerance range surrounding it and preferably to actuate a corresponding output as to whether the pressure measurement values or their time sequences are in a desired ratio with the respective threshold value and/or tolerance range.

All the features described above can naturally be combined with one another in any desired manner within the framework of the present invention.

Further advantages, features, and effects of the present invention result from the following description of an embodiment of the present invention with reference to the Figures.

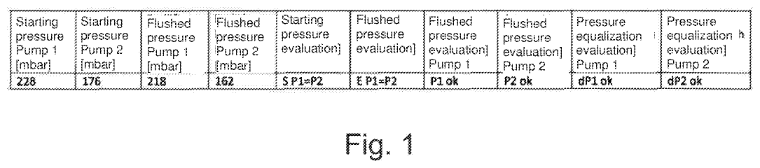

FIG. 1 shows by way of example an output of pressure measurement values and evaluation results in accordance with the present invention; and

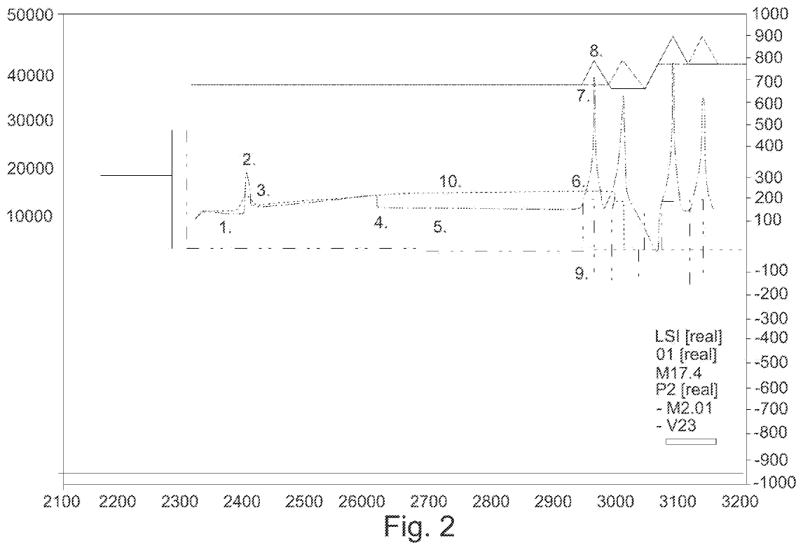

FIG. 2 shows an exemplary pressure sequence during a coupling procedure of a disposable to a dialysis machine that is based on pressure measurements at a plurality of times.

The starting pressures of the pumps 1 and 2 shown in FIG. 1 correspond to the chamber pressures of two pumps 1 and 2 that were measured at the time of the coupling procedure before the flushing of the disposable at the time marked by number 4 in FIG. 2.

The starting pressures of the pumps 1 and 2 shown in FIG. 1 correspond to the chamber pressures of the two pumps 1 and 2 that were measured at the time of the coupling procedure after the flushing of the disposable at the time marked by number 5 in FIG. 2.

The column “Starting pressure evaluation” reflects an evaluation of the synchronization or coordination of the chamber pressures of pump 1 and pump 2 before the flushing of the disposable. A check is made in the evaluation as to whether the starting pressures of pumps 1 and 2 are the same or do not exceed a maximum deviation from one another, e.g. 100 mbar.

The column “Flushed pressure evaluation” reflects an evaluation of the synchronization or coordination of the chamber pressures of pump 1 and pump 2 after the flushing of the disposable. A check is made in the evaluation as to whether the starting pressures of pumps 1 and 2 are the same or do not exceed a maximum deviation from one another, e.g. 100 mbar.

The column “Flushed pressure evaluation, pump 1” reflects an evaluation of the pressure level of pump 1 after the flushing of the disposable. A check is made in the evaluation as to whether the pressure is in a desired range and does not e.g. exceed a maximum value of 4000 mbar.

The column “Flushed pressure evaluation, pump 2” reflects an evaluation of the pressure level of pump 2 after the flushing of the disposable. A check is made in the evaluation as to whether the pressure is in a desired range and does not e.g. exceed a maximum value of 4000 mbar.

The column “Pressure equalization evaluation, pump 1” reflects an evaluation of the pressure drop in pump 1 after the flushing of the disposable. A check is made in the evaluation as to whether the pressure is in a desired range and does not e.g. fall below a minimum value of 10 mbar.

The column “Pressure equalization evaluation, pump 2” reflects an evaluation of the pressure drop in pump 2 after the flushing of the disposable. A check is made in the evaluation as to whether the pressure is in a desired range and does not e.g. fall below a minimum value of 10 mbar.

The evaluation of the pressure drop in pump 1 can, alternatively or additionally, be used to recognize an incorrectly reused (consumed) disposable and a corresponding error message can be output.

The typical working range for the coupling of a disposable moves in the range from 0 to 500 mbar.

An embodiment for the carrying out of the monitoring of the automatic setup procedure will be explained in the following with respect to FIG. 2. The monitoring of a coupling procedure by the carrying out of a method in accordance with the invention preferably takes place automatically, but can also be manually triggered or started.

In the example of FIG. 2, the disposable is a cassette system (also called a disposable cassette) that has a flexible membrane that spans a hard part.

The preparation of the machine first takes place before the start of a dialysis treatment. Various system tests (flushing, pressure check, determining the membrane tension of the pump dome at the machine side, etc.) are carried out for this purpose without an inserted cassette.

The disposable cassette is subsequently inserted and the working parameters (e.g. calibrating the membrane deflection in the cassette, setting the working range of the membrane pump at the machine side, etc.) are determined.

To ensure an exact balancing of the volumes to be pumped and thus patient safety or the achieving of a therapeutic goal, the disposable cassette has to be coupled to the machine block of the machine with an exact fit.

The check of the setup process and the ensuring of an exact coupling of the cassette in the machine now takes place such that pressure measurements are carried out at different times during the setup phase and statements on the quality of the setup process can be given or errors can be identified with reference to the pressure measurement profile over the time. A corresponding pressure measurement profile and its evaluation is shown in FIG. 2.

In FIG. 2, the numerals define times during a coupling procedure of a disposable to a dialysis machine at which a pressure measurement was carried out. The pressure can alternatively also be measured continuously during the total coupling procedure. In this respect, the numerals define the following pressure measurements or the following stages in the coupling procedure:
1. Hydraulic pressure before the coupling of the disposable article;
2. Contact dynamics and stretching of the disposable article during the coupling;
3. Counterpressure of the closed air in the disposable article;
4. Flushing of the disposable article to atmosphere after opening the patient valve;
5. Hydraulic pressure after the coupling of the disposable article;
6. Hydraulic pressure profile during the movement of the pump from the coupling position in the direction of the disposable article base;
7. Hydraulic pressure increase on reaching the disposable article base;
8. Length sensor position of the pump;
9. PWM control of the pump;
10. Positioning accuracy of the machine block with respect to disposable article.

The following conclusions can e.g. be made with respect to the quality of the coupling of the disposable to the blood treatment device and to the property of the disposable from the pressure measurements at the aforesaid times in the coupling procedure.

The pressure level at measurement point 1. indicates:
Too high a pressure means an incorrectly inserted disposable article that was incorrectly placed into the guide by the user.

The pressure level at measurement point 2. indicates:
No pressure peak means that a disposable article has not been inserted.
Too high a pressure means that the disposable article is not adjusted and does not center in the receptacle, e.g. a drawer or a door to receive the disposable article.

The pressure level at measurement point 3. indicates:
The difference of the pressure from measurement point 3. and 1. indicates an air cushion in the disposable article.
A lack of an air cushion indicates a leak in the disposable article.

The pressure level at measurement point 4. indicates:
A flushing of the disposable article to atmosphere takes place on the pressure drop at measurement point 4. and a closed patient line can be recognized.

The pressure level at measurement point 5. indicates:
The difference of the pressure from measurement points 1. and 5. shows the tension of the film of the disposable article.
A conclusion on a low temperature of the disposable article can be drawn by means of a high tension of the film.
A conclusion on a multiple use of the disposable article can be drawn by means of a low tension of the film.

The pressure level at measurement point 7. indicates:
The increase in pressure at measurement point 7. shows the reaching of a fixed abutment and the position value (measurement point 8.) of the disposable article. The tolerance of the disposable article can be looked at with these parameters.

The pressure level at measurement point 10. indicates:
The difference of the pressure levels between measurement points 10. to 5. shows the reaching of a fixed mechanical abutment. The positioning accuracy of the machine block with respect to the disposable article and the drawer can be derived from this pressure difference.

The following actions can be carried out on the basis of the aforesaid conclusions with respect to the quality of the coupling of the disposable to the blood treatment device and the properties of the disposable:
I. The coupling process can be carried out again if the detected quality of the coupling is insufficient.
II. User recommendations can be displayed that, for example, give the user action instructions to improve the quality of the coupling.
III. A qualitative classification of the disposable article coupling can be carried out; a school grade can be awarded, for example.
IV. Production tolerances of the machine design can be recognized.

At least the following technical advantages can thus be reached with the present invention.
a) Reducing setup errors
b) No waste of disposable articles
c) No discarding of dialyzate solution
d) Improved user recommendations, e.g.
a. Temperature of the disposable article too cold.
b. Disposable article incorrectly inserted.
c. Disposable article leaks.
d. Disposable article has been used more than once.
f) Recognizing quality tolerances in the production and manufacture of the machine components and of the disposable article.
g) Monitoring of the machine properties as part of predictive maintenance.

1. A blood treatment device, preferably a dialysis machine, that is configured to be used with a disposable, preferably with a cassette system, and that has a coupling region for the disposable and a control unit that is configured to carry out a plurality of pressure measurements at different times during a coupling procedure of the disposable to the blood treatment device, with a quality of the coupling of the disposable to the blood treatment device being able to be determined with reference thereto.

2. A blood treatment device in accordance with claim 1, furthermore having an evaluation unit that is configured to evaluate a time sequence of a pressure profile prepared on the basis of the plurality of pressure measurements, preferably with respect to a pressure level and/or to a pressure change rate and to determine the quality of the coupling of the disposable to the blood treatment device on the basis thereof.

3. A blood treatment device in accordance with claim 1, characterized in that environmental conditions and/or properties of the disposable, in particular material properties and/or tolerances are taken into account in the determination of the quality of the coupling of the disposable to the blood treatment device.

4. A blood treatment device in accordance with claim 1, characterized in that the blood treatment device has one or more pumps; and in that the plurality of pressure measurements preferably take place at or in the one or more pumps.

5. A blood treatment device in accordance with claim 1, further comprising at least one pressure measurement device that is configured to detect a pressure in the disposable, in at least one pump, and/or in at least one line.

6. A blood treatment device in accordance with claim 1, characterized in that the control device is configured to carry out a pressure measurement at least a first time after the disposable has been mounted on the blood treatment device and before the disposable has been flushed and/or at a second time after the disposable has been installed on the blood treatment device and after the disposable has been flushed.

7. A blood treatment device in accordance with claim 1, characterized in that the evaluation unit is configured to compare the pressure measurement values determined for a plurality of pumps or their time sequences with one another respectively to determine the quality of a coordination of the pumps.

8. A blood treatment device in accordance with claim 1, characterized in that the evaluation unit is configured to compare the determined pressure measurement values and/or their time sequences in each case with a threshold value and/or with a tolerance range surrounding it; and in that the blood treatment device is preferably furthermore configured to actuate a corresponding output as to whether the pressure measurement values or their time sequences are in a desired ratio with the respective threshold value and/or tolerance range.

9. A blood treatment device in accordance with claim 1, characterized in that, the blood treatment device is configured to automatically electronically register and document the detected pressure measurement values and the data prepared by the evaluation unit.

10. A blood treatment device in accordance with claim 1, characterized in that the blood treatment device is configured to actuate an output with respect to the determined quality of the coupling of the disposable to the blood treatment device and/or to only release an operation of the blood treatment device when a certain quality of the coupling of the disposable to the blood treatment device exceeds a predetermined threshold value.

11. A method of determining a quality of the coupling of a disposable, in particular a cassette system, to a blood treatment device, in particular a dialysis machine, the methods comprising the steps:
carrying out a plurality of pressure measurements at different times during a coupling procedure of the disposable to the blood treatment device to determine pressure measurement values; and
evaluating a time sequence of a pressure profile prepared on the basis of the plurality of pressure measurements, preferably with respect to a pressure level and/or a pressure change rate and determining the quality of the coupling of the disposable to the blood treatment device on the basis thereof.

12. A method in accordance with claim 11, characterized in that a pressure measurement is at least carried out at a first time after the disposable has been installed on the blood treatment device and before the disposable has been flushed and/or at a second time after the disposable has been installed on the blood treatment device and after the disposable has been flushed.

13. A method in accordance with claim 11, characterized in that the plurality of pressure measurements take place at or in the one or more pumps of a blood treatment device, preferably a blood treatment device in accordance with one of the claims 1 to 10.

14. A method in accordance with claim 11, characterized in that pressure measurement values are determined for a plurality of pumps and that the pressure measurement values or their time sequences are respectively compared with one another to determine the quality of a coordination of the pumps.

15. A method in accordance with claim 11, characterized in that the determined pressure measurement values or their time sequences are respectively compared with a threshold value and/or with a tolerance range surrounding it and preferably to actuate a corresponding output as to whether the pressure measurement values or their time sequences are in a desired ratio with the respective threshold value and/or tolerance range.
